(19) United States
(12) Patent Application Publication
(10) Pub. No.: US 2025/0387080 A1
(43) Pub. Date: Dec. 25, 2025

(54) ESTIMATING RESPONSIVENESS TO DENERVATION THERAPY

(71) Applicant: Medtronic Ireland Manufacturing Unlimited Company, Dublin (IE)

(72) Inventors: Douglas A. Hettrick, Hugo, MN (US); Martin Fahy, Brooklyn, NY (US); Carlos F. Haro, New York, NY (US); Heidrun Behrmann, Frankfurt (DE); Julie Trudel, Santa Rosa, CA (US)

(21) Appl. No.: 19/239,082

(22) Filed: Jun. 16, 2025

Related U.S. Application Data
(60) Provisional application No. 63/662,702, filed on Jun. 21, 2024.

(51) Int. Cl. A61B 5/00; A61B 5/021

(52) U.S. Cl. CPC A61B 5/4848; A61B 5/021; A61B 5/7275

(57) ABSTRACT

An example computing system includes a memory and one or more processors coupled to the memory. The one or more processors obtain values for a plurality of patient parameters of a patient with hypertension that relate to a physiological condition of the patient. Before delivery of denervation therapy to the patient, the one or more processors determine, using a computational model, a predicted change in blood pressure of the patient if the denervation therapy is delivered based on the values of the plurality of patient parameters based on the values of the plurality of patient parameters. The one or more processors output, responsive to determining the predicted change in blood pressure of the patient, an indication associated with the predicted change in blood pressure of the patient.

This application claims the benefit of and priority to U.S. Provisional Application No. 63/662,702, filed Jun. 21, 2024, the entire disclosure of which is incorporated by reference herein.

### TECHNICAL FIELD

This disclosure generally relates to estimating responsiveness to denervation therapy.

### BACKGROUND

Overstimulated or excessively active nerves may result in adverse effects to organs or tissue served by the respective nerves. For example, for some patients, heart, circulatory, or renal disease may be associated with pronounced cardio-renal sympathetic nerve hyperactivity. Stimulation of the renal sympathetic nerves can cause one or more of an increased renin release, increased sodium (Na⁺) reabsorption, or a reduction of renal blood flow. The kidneys may be damaged by direct renal toxicity from the release of sympathetic neurotransmitters (such as norepinephrine) in the kidneys in response to high renal nerve stimulation. Additionally, the increase in release of renin may ultimately increase systemic vasoconstriction, aggravating hypertension.

Percutaneous renal denervation is a procedure that can be used for treating hypertension. During a renal denervation procedure, a clinician delivers stimuli or energy, such as radiofrequency, ultrasound, microwave, gamma, cooling, drug injection, or other modality, to a treatment site to reduce activity of nerves surrounding a blood vessel. The stimuli or energy delivered to the treatment site may provide various therapeutic effects through alteration of sympathetic nerve activity. Percutaneous denervation of the afferent and efferent renal nerves may result in a reduction in blood pressure (BP) in some patients with uncontrolled hypertension. However, not all patients experience a reduction in BP immediately or over time following the renal denervation (RDN) procedure, as some patients are “non-responders.” Even among responders, an extent of the reduction of BD may vary substantially.

### SUMMARY

In general, the disclosure describes a non-invasive, accurate, and reliable predictor to quantify, before performing an RDN procedure to deliver denervation therapy, a response of a patient to denervation therapy, and/or illustrate potential benefits or other considerations of the RDN procedure once the patient has been identified. Denervation therapy may provide a therapeutic benefit to certain patients, such as mitigation of symptoms associated with renal sympathetic nerve overactivity. However, not all patients respond well to denervation therapy, such as patients with decreased basal sympathetic nervous system activity.

Computer-based predictors described herein may help predict a potential blood pressure reduction before performing an invasive denervation procedure that may end up being ineffective or less effective on balance of other factors, which may improve health care efficiency and patient care by reducing performance of less effective procedures while also reducing patient discomfort in undergoing a less effective procedure. A computing device uses a model to predict a predicted reduction in blood pressure of a patient with hypertension after denervation therapy. The model uses various patient parameters of the patient that relate to a physiological condition and can be input by the patient or a clinician of the patient, or automatically tracked using an external device. The computing device further modifies the patient parameters using weighting coefficients that are based on physiological data, such as blood pressure data, from large, multivariable patient populations, and that may be updated over time based on new blood pressure data or additional patient characteristics that may affect the reduction in blood pressure. The resulting quantified output includes an indication associated with the predicted blood pressure of the patient that can assist the patient or the clinician of the patient in assessing potential benefits of the denervation therapy against other considerations.

In one example, this disclosure is directed to a computing device that includes a memory and one or more processors coupled to the memory. The one or more processors are configured to obtain values for a plurality of patient parameters of a patient with hypertension. Each patient parameter of the plurality of patient parameters relates to a physiological condition of the patient. The one or more processors are configured to before delivery of denervation therapy to the patient, determine, using a computational model, a predicted change in blood pressure of the patient if the denervation therapy is delivered based on the values of the plurality of patient parameters. Each patient parameter of the plurality of patient parameters is modified by a weighting coefficient of a plurality of weighting coefficients of the computational model. The one or more processors are configured to output, responsive to determining the predicted change in blood pressure of the patient, an indication associated with the predicted change in blood pressure of the patient.

In another example, this disclosure is directed to a method, by a computing system, for predicting a change in blood pressure of a patient with hypertension. The method includes obtaining values for a plurality of patient parameters of the patient with hypertension. Each patient parameter of the plurality of patient parameters relates to a physiological condition of the patient. The method includes before delivery of denervation therapy to the patient, determining, using a computational model, a predicted change in blood pressure of the patient if the denervation therapy is delivered based on the values of the plurality of patient parameters. Each patient parameter of the plurality of patient parameters is modified by a weighting coefficient of a plurality of weighting coefficients of the computational model. The method includes outputting, by the computing system and responsive to determining the predicted change in blood pressure of the patient, an indication associated with the predicted change in blood pressure of the patient.

Further details of one or more examples of this disclosure are set forth in the accompanying drawings and in the description below. Other features, objects, and advantages will be apparent from the description and drawings, and from the claims.

The above summary is not intended to describe each illustrated example or every implementation of the present disclosure.

### BRIEF DESCRIPTION OF DRAWINGS

FIG. 1 is a conceptual diagram illustrating an example system for estimating a response to denervation therapy, in accordance with some examples of the current disclosure.

FIG. 2 is a block diagram illustrating an example configuration of a computing system configured to estimate a response to denervation therapy, in accordance with some examples of the current disclosure.

FIG. 3A is a flow diagram illustrating an example technique for estimating a response to denervation therapy, in accordance with some examples of the current disclosure.

FIG. 3B is a flow diagram illustrating an example technique for generating a computational model for estimating a response to denervation therapy, in accordance with some examples of the current disclosure.

FIG. 4A is a diagram of an example user interface for receiving patient parameters and outputting an indication of an estimate of a response to denervation therapy, in accordance with some examples of the current disclosure.

FIG. 4B is a graph of an output of the example user interface of FIG. 4A, in accordance with some examples of the current disclosure.

FIG. 4C is a diagram of an example user interface for receiving patient parameters for three different cases and outputting an indication of an estimate of a response to denervation therapy for each of the three cases, in accordance with some examples of the current disclosure.

FIG. 4D is a graph of an output of the example user interface of FIG. 4C, in accordance with some examples of the current disclosure.

### DETAILED DESCRIPTION

Denervation therapy, such as renal denervation (RDN) therapy, may be used to render a nerve inert, inactive, or otherwise completely or partially reduced in function, such as by ablation or lesioning of the nerve. Denervating an overactive nerve may provide a therapeutic benefit to a patient. For example, renal denervation may mitigate symptoms associated with renal sympathetic nerve overactivity, such as hypertension. Denervation therapy may include delivering electrical and/or thermal energy to a target nerve, and/or delivering a chemical agent to a target nerve. In the case of renal denervation therapy, the denervation energy or chemical agents can be delivered, for example, via a therapy delivery device (e.g., a catheter) disposed in a blood vessel (e.g., the renal artery) proximate to the renal nerve.

The renal sympathetic nervous system has been identified as a potential major contributor to the complex pathophysiology of hypertension, or elevated systemic blood pressure (BP). Renal denervation may reduce renal sympathetic nerve overactivity and cause a reduction in systemic BP as a treatment for hypertension. In some patients, renal denervation may reduce systolic BP in a range of approximately 5 millimeters of mercury (mmHg) to 30 mmHg. Denervation therapy may be used as treatment for other ailments associated with changes in sympathetic nervous system activity as well, such as arrhythmias and heart failure.

While RDN therapy may provide a reduction in systemic BP for a large population of patients with hypertension, a response to renal denervation may vary substantially between patients due to various physiological characteristics of the patient. For example, only about two thirds of patients with hypertension may experience a reduction in blood pressure greater than 5 mmHg within the first several months after RDN therapy. Certain measurable patient characteristics related to potentially higher baseline sympathetic activity, such as increased heart rate, increased plasma renin levels, increased muscle sympathetic nerve activity, or increased renal norepinephrine spillover, and/or potentially lower basal arterial stiffness, such as may be associated with a capacity for additional peripheral vasodilation, may indicate a higher responsiveness to RDN. However, the effect of these patient characteristics may not be quantified in a way that can be assessed against other considerations, such as other therapies. The uncertainty of response can be difficult for both clinicians and patients as they struggle to communicate and understand, respectively, the potential factors, including benefits, of the RDN procedure.

The present disclosure describes various aspects of techniques related to predicting a potential response, such as a reduction of blood pressure, of a patient to denervation therapy. While the above examples discuss renal denervation responsiveness in reducing hypertension, the techniques described herein may similarly be used to predict renal denervation responsiveness for treatment of other ailments associated with changes in sympathetic nervous system activity, such as arrhythmias and heart failure. More generally, the techniques described herein may be used to predict responsiveness for treatment of an ailment that may be influenced by a variety of patient characteristics, and for which a model may be based on indicator data (e.g., blood pressure data) of a large patient population that includes the patient characteristics.

In this way, the example techniques improve the technology of RDN therapy effectiveness prediction by integrating in a practical application the computational model based determination of a predicted change in blood pressure of the patient if denervation therapy were delivered, but before delivery of the denervation therapy. For instance, as described in more detail, the model generation and computations associated with predicting change in blood pressure of the patient may not be possible by the clinician for accurate prediction. With the example techniques described in this disclosure, a computing system configured to determine, using a computation model, a predicted change in blood pressure of the patient may provide a more accurate prediction as compared to other computing system that do not perform the example techniques or clinician generated prediction.

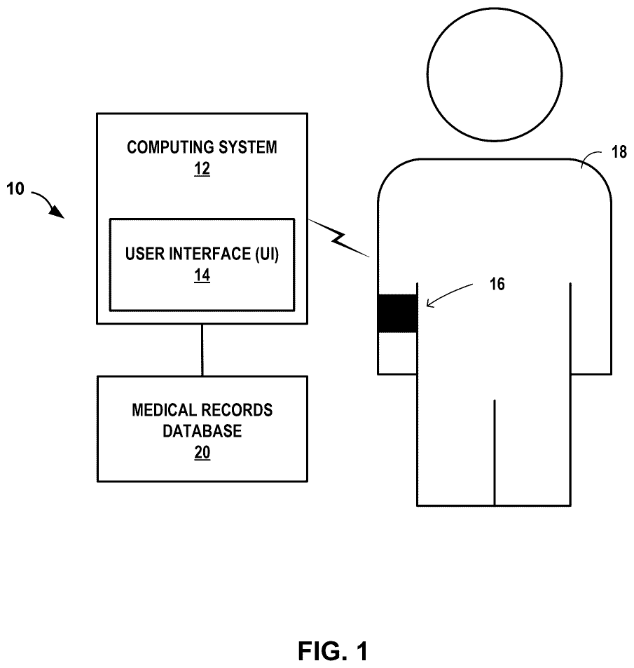

FIG. 1 is a conceptual diagram illustrating an example system 10 for estimating a response of a patient 18 to denervation therapy. As shown in FIG. 1, system 10 includes a computing system 12, and optionally, a physiological sensor device 16 and/or a medical records database 20. Computing system 12 may include one or more computing devices used in a home, ambulatory, clinic, or hospital setting. Computing system 12 may include, for example, a clinician programmer, a desktop computer, a laptop computer, a workstation, a server, a mainframe, a cloud computing system, a smartphone, combinations thereof, or the like.

Computing system 12 may be configured to, via a user interface device 14 (“UI 14”), receive input data from a user, such as patient 18 or a clinician of patient 18, output information to the user, or both. In some examples, UI 14 may include a display (e.g., a liquid crystal display (LCD) or light emitting diode (LED) display), such as a touch-sensitive display; one or more buttons; one or more keys (e.g., a keyboard); a mouse; one or more dials; one or more switches; a speaker; one or more lights; combinations thereof; or the like.

In some examples, computing system 12 may be communicatively coupled to a physiological sensor device 16. Physiological sensor device 16 may be a short-term device, such as a blood pressure sensor, or a long-term device, such as a wearable medical, health, or fitness tracking device, configured to measure physiological signals of patient 18 as sensor data. Some examples of physiological signals that may be sensed by such devices may include blood pressure, electrocardiogram (ECG) signals, respiration signals, perfusion signals, activity and/or posture signals, pressure signals, blood oxygen saturation signals, body composition, fluid impedance signals, and blood glucose or other blood constituent signals. Physiological sensor device 16 may be configured to collect and/or communicate the sensed physiological signals and/or data based on the sensed physiological signals to computing system 12. For example, physiological sensor device 16 may detect blood pressure values of patient 18 and collect and/or communicate the detected blood pressure values with computing system 12.

In some examples, computing system 12 may be communicatively coupled to a medical records database 20. Medical records database 20 may be configured to store medical records data 22 associated with patient 18, such as previously-obtained physiological data, procedural history, or any other data associated with patient 18 that may indicate one or more physiological conditions that may influence an effectiveness of denervation therapy for patient 18. Computing system 12 may query medical records database 20 for the medical records data.

As mentioned above, an effectiveness of denervation therapy may vary among patients due to physiological differences between patients. When deciding whether to proceed with denervation therapy, patient 18, in consultation with a clinician, may evaluate benefits of, risks of, and alternatives to denervation therapy. In accordance with techniques described herein, computing system 12 is configured to predict a response of patient 18 to denervation therapy, such as a change in blood pressure. Computing system 12 may receive values for a plurality of patient parameters of patient 18. Each patient parameter relates to a physiological condition of the patient, such as a physiological measurement or a medical history event. Values of the patient parameters may include input data received via UI 14, sensor data received from physiological sensor device 16, and/or medical records data received from medical records database 20.

Prior to delivering denervation therapy, computing system 12 may determine, using a computational model, a predicted blood pressure of patient 18 if denervation therapy is delivered based on the plurality of patient parameters of patient 18. The computational model may be developed from population data for a population of patients with hypertension, and may account for both fixed effects (i.e., variables that have a similar effect on blood pressure across patients) and random effects (i.e., variables that have random effects on blood pressure due to personal or environmental variation). The computational model may include any computational model that receives patient parameters as inputs and generates a predicted blood pressure based on parameters derived from a population of patients having undergone denervation therapy including, but not limited to, regression models, Bayesian models, binary decision tree models, neural network models, and/or models derived using artificial intelligence or machine learning techniques. In some examples, the computational model includes a plurality of weighting coefficients derived from the population data. Each patient parameter is modified by a weighting coefficient of the computational model, and the computational model can be updated with both new patient parameters and updated values of weighting coefficients as significant patient parameters are identified and their relative influence quantified. The computational model may further account for changes in blood pressure over time as a result of the denervation therapy. The predicted change in blood pressure may include a reduction in blood pressure or, if based on a baseline blood pressure prior to denervation, an absolute blood pressure of patient 18.

In response to determining the predicted blood pressure of patient 18, computing system 12 may output an indication associated with the predicted blood pressure of patient 18. The indication may provide context for the change in blood pressure, such as through a visual indication (e.g., a graph) or a relative indication (e.g., comparison to actual or hypothetical patients having different values of patient parameters). As a result, computing system 12 may provide a non-invasive, accurate, and reliable predictor to quantify, before performing an RDN procedure to deliver denervation therapy, a potential benefit of denervation therapy to patient 18. Such prediction may aid patient 18 and the clinician of patient 18 in evaluating potential benefits of denervation, particularly on a reduction in blood pressure or other hemodynamic parameter over time.

While computing system 12 has been described with respect to a predicted blood pressure, computing system 12 may be additionally or alternatively configured to predict other conditions using a computational model. For example, the computational model may be based on population data that includes parameters related to cardiovascular risk, such as myocardial infarction, or renal failure. The computational model may receive patient parameters as inputs and generate a prediction of the cardiovascular risk based on the patient parameters.

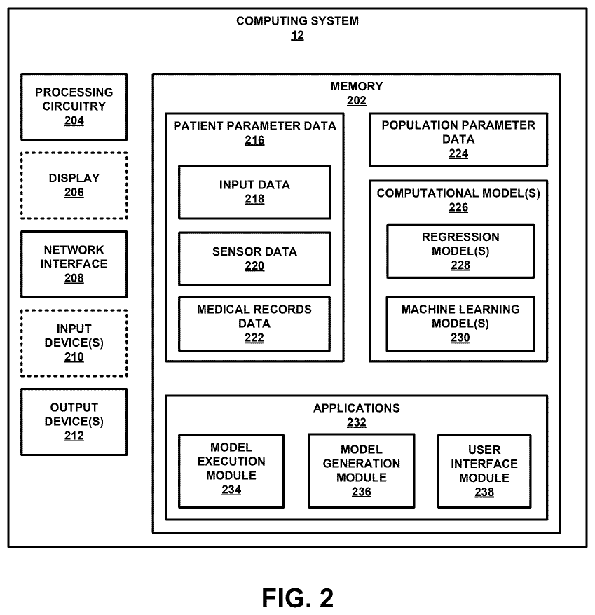

FIG. 2 is a block diagram illustrating an example configuration of computing system 12 of FIG. 1 configured to estimate a response of a patient to denervation therapy. Computing system 12 may include a workstation, a desktop computer, a laptop computer, a smart phone, a tablet, a dedicated computing device, or any other computing device capable of performing the techniques of this disclosure. In the example of FIG. 2, computing system 12 includes a memory 202, processing circuitry 204, a display 206, a network interface 208, an input device(s) 210, and an output device(s) 212, each of which may represent any of multiple instances of such a device within the computing system, for ease of description. For example, while computing system 12 is illustrated as a single computing device, computing system 12 may include a first computing device having first set of processors configured to perform a first application in memory 202, and a second computing device having a second set of processors configured to perform a second application in memory 202.

Display 206 may be configured to display information to a user, such as a patient or a clinician. Display 206 may be touch sensitive or voice activated (e.g., via one or more sensors which may include one or more microphones), enabling display 206 to serve as both an input and output device. Alternatively, a keyboard (not shown), mouse (not shown), or other data input devices (e.g., input device(s) 210) may be employed. Input device(s) 210 may include any device that enables a user to interact with computing system 12, such as, for example, a mouse, keyboard, foot pedal, touch screen, augmented-reality input device receiving inputs such as hand gestures or body movements, or voice interface. Output device(s) 212 may include any connectivity port or bus, such as, for example, parallel ports, serial ports, universal serial busses (USB), or any other similar connectivity port known to those skilled in the art. Network interface 208 may be adapted to connect to a network, such as a local area network (LAN) that includes a wired network or a wireless network, a wide area network (WAN), a wireless mobile network, a Bluetooth network, or the internet. Computing system 12 may receive updates to its software, for example, applications 232, via network interface 208.

Memory 202 of computing system 12 includes any non-transitory computer-readable storage media for storing data or software that is executable by processing circuitry 204 and that controls the operation of computing system 12. In one or more examples, memory 202 may include one or more solid-state storage devices, such as flash memory chips, or one or more mass storage devices connected to processing circuitry 204 through a mass storage controller (not shown) and a communications bus (not shown). Memory 202 may be configured to store patient parameter data 216, population parameter data 224, computational model(s) 226, and applications 232.

Patient parameter data 216 may include any data related to a patient, including data indicating a physiological condition of the patient that may influence an effectiveness of denervation on blood pressure. As will be described further below regarding model generation, patient parameters may be identified from significant fixed effects of population parameter data 224, and patient parameter data 216 may include values that correspond to these patient parameters. Patient parameters may include any of patient demographic characteristics, patient ambulatory blood pressure monitoring (ABPM) and/or patient home blood pressure monitoring (HBPM) characteristics, patient imaging characteristics, patient physiological characteristics, patient procedural and medication history, and other measurable or estimable patient characteristics that may influence an effectiveness of denervation therapy on blood pressure.

Patient demographic characteristics may include, but are not limited to, age, sex, or race. Patient ABPM and/or HBPM characteristics may include, but are not limited to, blood pressure characteristics, such as baseline blood pressure, resting blood pressure, stressed blood pressure, nighttime blood pressure, or blood pressure variability (diurnal/dipping/morning surge, day to day/week to week, or smoothness); heart rate characteristics, such as resting heart rate or heart rate variability; and other characteristics that may be measured or quantified using ABPM and/or HBPM. Patient imaging characteristics may include, but are not limited to, characteristics determined through imaging/neuro-imaging, such as mIBG, F(18), or aortic calcification; characteristics indicated by anatomy, such as accessory arteries present/treatable or renal artery diameter; or other characteristics that may be visually indicated using images of an anatomy of a patient.

Patient physiological characteristics may include, but are limited to, body mass index (BMI); arterial stiffness, such as indicated by pulse wave velocity, pulse pressure, diastolic blood pressure, or central arterial pressure; baroreceptor sensitivity; galvanic skin response; stress response, such as indicated by cold pressor (BP response) or mental stress/Stroop color test; quantitative pupillography; drug response, such as clonidine response or captopril response; baseline estimated glomerular filtration rate (eGFR) for chronic kidney disease (CKD); baseline serum creatinine; blood biomarkers, such as renin, S100, neuropeptide Y, tyrosine hydroxylase, or NE; urine biomarkers, such as inflammatory markers, NE, or non-adherence to medications; hemodynamics (e.g., renal artery stiffness); or other measurable physiological characteristics.

Patient procedural and medication history may include, but is not limited to, presence of co-morbidities, such as myocardial infarction, type 2 diabetes, smoking, heart failure, or atrial fibrillation; presence of sleep disorders, such as sleep apnea; presence of certain prescribed medication classes, such as antihypertensive medications (e.g. beta blockers); number of certain prescribed medication classes; procedure characteristics, such as procedure duration, catheter time, contrast volume, and number of ablations; or other conditions in medical history that may influence and/or be correlated with an effectiveness of a response of denervation therapy to blood pressure.

Patient parameter data 216 may include data from a variety of sources, which may include input data 218, sensor data 220, and medical records data 222. Input data 218 may be obtained by computing system 12 via input device 210. Input data 218 may include any values of patient parameters input by a user. For example, a patient or clinician may enter values of one or more patient parameters directly into input device 210, such as during a patient interview or patient diagnostic testing. Sensor data 220 may be generated by physiological sensor device 16 and obtained via network interface 208 or another device interface, which may be communicatively coupled to physiological sensor device 16. Sensor data 220 may include any physiological data, such as heart rate data, of the patient obtained in real-time or near real-time. For example, physiological sensor device 16 of FIG. 1 worn by the patient may send physiological sensor data, such as blood pressure measurements or heart rate measurements, to computing system 12. Medical records data 222 may be obtained by computing system 12 via network interface 208, which may be communicatively coupled to medical records database 20 of FIG. 1. Medical records data 222 may include any physiological data or medical history data of the patient obtained previously. For example, medical records database 20 of FIG. 1 may send medical records data 222 relating to the patient parameters to computing system 12 in response to a query.

In some examples, sensor data 220 may be recorded in a natural (i.e., unperturbed) state, or in response to natural (e.g. Detected sleep apneic event) or known artificial perturbations such as Valsalva maneuver, Mueller maneuver, orthostasis (e.g., standing from seated position), cold pressor, mental stress (e.g. mental math, Stroop color test, etc.). Such acute responses may be quantified and entered separately into the computational model. For example, computing device 12 may convert sensor data 220 into a particular value for input into the computational model.

As mentioned above, computing system 12 is configured to estimate a change in blood pressure based on a computational model that is derived from data for a population of patients with hypertension that have undergone denervation therapy. Population parameter data 224 may include any data that may indicate a physiological condition of a particular patient that may have influenced an effectiveness of denervation, such as a change in blood pressure as a result of denervation therapy. Population parameter data 224 may include medical records data for each patient in the population, which may include a value for at least one fixed effect and at least one blood pressure measurement. Fixed effects may include any variable of the patient of the population, including variables that may not be significant enough to be captured in the plurality of patient parameters of patient parameter data 216. Fixed effects may include any of the patient parameters described above with respect to patient parameter data 216, including patient demographic characteristics, patient ambulatory blood pressure monitoring (ABPM) and/or home blood pressure monitoring (HBPM) characteristics, patient imaging characteristics, patient physiological characteristics, patient procedural and medication history, and other measurable or estimable patient characteristics that may influence an effectiveness of denervation therapy on blood pressure.

In some examples, population parameter data 224 may be heterogeneous multivariate data for which data entries may include incomplete data for one or more fixed effects. Medical records data may include data that was captured at different times and in different clinical settings, such that the variables for which data is available for two or more patient entries may be different. For example, a first medical record entry for a first patient obtained during a first clinical trial may include values for a first set of fixed effects (e.g., stress response) and a first blood pressure measurement at a first time (e.g., 3 months follow-up), while a second medical record entry for a second patient obtained during a second clinical trial may include a second set of fixed effects (e.g., heart rate variability), different from the first set of fixed effects, and a second blood pressure reading at a second time (e.g., 6 months follow-up), different from the first time.

Population parameter data 224 may be obtained by computing system 12 via network interface 208, which may be communicatively coupled to medical records database 20. Population parameter data 224 may be updated, such as periodically or in real-time. For example, new clinical trial data may be added to population parameter data 224, such as medical records data that includes new fixed effects for which computational model 226 may be based, or previous fixed effects which may be newly recognized as significant.

Computational model 226 may include any computational model that can be generated from population parameter data 224 and used to predict a response to denervation therapy, such as a change in blood pressure, based on patient parameter data 216. As will be described further below, when executing computational model 226, model execution module 234 receives as input the values of patient parameters from patient parameter data 216. In some examples, such as examples in which computational model 226 is a regression model, computational model 226 includes a plurality of weighting coefficients, and may also include one or more bias (or intercept) coefficients or other coefficients representing random effects. To execute computational model 226, model execution module 234 modifies (e.g., multiplies) each patient parameter by a weighting coefficient of computational model 226 to normalize the patient parameter and account for an influence of the patient parameter on the predicted change in blood pressure. Computational model 226 generates as output the predicted change in blood pressure, such as by summing the products of the patient parameters and weighting coefficients, as well as any bias coefficients or variables related to random effects.

In some examples, computational model 226 includes a regression model 228. As will be described further with respect to model generation module 236, regression model 228 may be derived from population parameter data 224 using one or more regression techniques. Regression model 228 may have the following general form:

yₜ=B₀+B₁x₁+B₂x₂+ . . . +Bₚxₚ+zₜ [Equation1]

In Equation 1 above, yₜ represents a reduction in blood pressure at a timepoint t, B₀ represents a bias coefficient (or intercept), B₁ represents a first weighting coefficient, x₁ represents a first patient parameter, B₂ represents a second weighting coefficient, x₂ represents a second patient parameter, Bₚ represents a last weighting coefficient, and xₚ represents a last patient parameter, and zₜ represents a weighting coefficient for follow-up blood pressure reduction at the timepoint t.

In the example above, Equation 1 may accommodate predictions of reduction of blood pressure at multiple timepoints. For example, a patient that undergoes denervation therapy may experience a gradual reduction in blood pressure due to physiological changes that result from the denervation therapy, such that subsequent follow-up blood pressure measurements may show a continued decrease in blood pressure. Typical timepoints may include 3 months, 6 months, 12 months, 24 months, and 36 months.

Regression model 228 may be a mixed multivariate linear regression model that incorporates both fixed effects and random effects. Fixed effects are variables or factors that have a specific, consistent impact on the dependent variable. These effects are considered to be constant and non-random across different observations, such as such as age or genders. The coefficients for fixed effects are estimated as part of the model, and apply uniformly to all individuals or units in the study. Fixed effects may include any of the patient parameters described above with respect to patient parameter data 216. Random effects, on the other hand, are variables or factors that introduce variability into the model that is specific to certain groups or clusters within the data, such as different measurements for a same patient, different patients within a same trial, or different trials. Random effects may not be needed in all examples, but may be included to account for the hierarchical or grouped structure of the data. Random effects may include, but are not limited to, different trials, different patients within a trial, and different blood pressure measurements for a patient.

In some examples, computational model 226 includes a machine learning model 230. As will be described further with respect to model generation module 236, regression model 228 may be derived from population parameter data 224 using one or more machine learning techniques. Machine learning model 230 may include, but is not limited to, Bayesian models, decision tree models, random forest models, support vector machines (SVM) models, and neural network models. As one example, machine learning model 230 may include a Bayesian model that is based on Bayes' Theorem, which relates current probability to prior probability and likelihood. The Bayesian model may incorporate patient parameter data 224 and update outcomes with evidence to offer a probabilistic interpretation of predictions. As another example, machine learning model 230 may include a decision tree model that uses a tree structure in which decisions are made at each node based on feature values, and in which the decision tree model splits population parameter data 224 into subsets based on feature value tests. As another example, machine learning model 230 may include a neural network model that includes layers of interconnected nodes that transform population parameter data 224 through learned weights.

Applications 232 may be one or more software programs stored in memory 202 and executed by processing circuitry 204 of computing system 12. Applications 232 of memory 202 may include a model execution module 234, a model generation module 236, and a user interface module 238, each of which may be executed by processing circuitry 204. For simplicity, functions performed, or configured to be performed, by applications 232 will be understood to be performed, or configured to be performed, by processing circuitry 204 according to a set of instructions. In some examples, different processors in processing circuitry 204 may perform instructions for different applications 232. For example, a first set of processors in processing circuitry 204, such as housed in a first computing device, may perform instructions for model execution module 234 and user interface module 238, while a second set of processors in processing circuitry 204, such as housed in a second computing device, may perform instruction for model generation module 236, as such applications 232 may be executed at substantially different times.

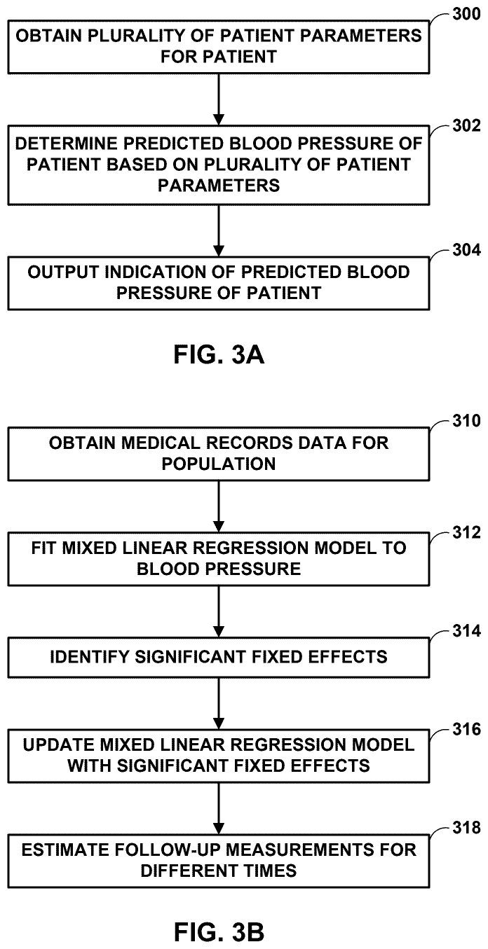

Model execution module 234 is configured to execute computational model 226 using patient parameter data 216 to determine a predicted change in blood pressure. FIG. 3A is a flow diagram illustrating an example technique for estimating a response to denervation therapy, in accordance with some examples of the current disclosure, and will be described with respect to model execution module 234.

Model execution module 234 may obtain values for a plurality of patient parameters of a patient with hypertension (300). As described in FIG. 2 above, each patient parameter of the plurality of patient parameters relates to a physiological condition of the patient, such as a physiological measurement or a medical history event. To obtain the values of the plurality of patient parameters, model execution module 234 may access patient parameter data 216 received from a variety of sources. For example, model execution module 234 may receive, via a user interface on input device 210, a value for at least one patient parameter, such as from input data 218; receive, from a medical records database, a value for at least one patient parameter, such as from medical records data 222; and/or receive, from a physiological sensor device, a value for at least one patient parameter, such as from sensor data 220.

Prior to delivering denervation therapy, model execution module 234 may determine, using computational model 226, a predicted change in blood pressure of the patient if the denervation therapy is delivered based on the values of the plurality of patient parameters (302). Each patient parameter of the plurality of patient parameters is modified by a weighting coefficient of a plurality of weighting coefficients of the computational model. For example, model execution module 234 may multiply each patient parameter by a corresponding weighting coefficient, and subsequently sum the values of these products with other values derived from population parameter data 224, such as values for bias coefficients and/or coefficients for random effects, such as a blood pressure reduction at different timepoints after denervation therapy. In some examples, computational model 226 may be a mixed multivariate linear regression model, such as described in FIG. 2 above. The determined change in blood pressure may include any of office and ambulatory systolic and diastolic blood pressure.

Model execution module 234 may output, responsive to determining the predicted change in blood pressure of the patient, an indication associated with the predicted change in blood pressure of the patient (304). The indication predicted change in blood pressure may include the absolute change in blood pressure or a relative change in blood pressure (such as compared to a baseline blood pressure), and may include any visual, audio, digital, or other indication.

In some examples, the indication associated with the predicted change in blood pressure may include a reference to a threshold. For example, a particular threshold that corresponds to a reduction in blood pressure (or corresponding reduction in cardiovascular risk) may be associated with a therapeutic benefit outweighing an age-based or other risk of denervation therapy, or may be associated with a therapeutic benefit substantial for a particular level of financial coverage by a third-party. Model execution module 234 may determine whether the predicted change in blood pressure exceeds a denervation therapy threshold, and based on whether the predicted change in blood pressure exceeds the denervation therapy threshold, whether the patient is or is not a candidate for the denervation therapy. For example, model execution module 234 may determine that the predicted change in blood pressure exceeds the threshold, and indicate that the patient likely is a candidate for the denervation therapy; on the other hand, model execution module 234 may determine that the predicted change in blood pressure does not exceed the threshold, and indicate that the patient likely is not a candidate for the denervation therapy. Model execution module 234 may output whether the patient is or is not a candidate for the denervation therapy, such as via output device 212. As such, model execution module 234 may provide an indication that incorporates other substantive metrics relevant to denervation therapy in a manner that is easy to understand.

In some examples, the indication associated with the predicted change in blood pressure may include a reference to a set of individuals or a population. For example, an average or typical reduction in blood pressure for a set of individuals or a population may be indicated for comparison with the predicted reduction in blood pressure. The set of individuals or the population may be selected from population parameter data 224 based on certain risks of denervation therapy, such as due to demographics or comorbidities, that may be shared with the patient.

In some examples, the indication associated with the predicted change in blood pressure may include identification of one or more patient parameters for which a current or future change may affect the predicted change in blood pressure in response to denervation therapy. For example, certain patient parameters may have a greater effect on a change in blood pressure after denervation therapy than other patient parameters. In such examples, model execution module 234 may determine a proportion of a change in blood pressure that may be attributable to one or more patient parameters, and indicate the proportion of the change, rank the patient parameters according to the proportion of the change, or filter the patient parameters according to a proportion of the change that exceeds a threshold for significance. In some instances, further changing one or more patient parameters, such as by lifestyle changes or future interventions, may predict a further change in blood pressure. In such examples, model execution module 234 may determine a first predicted change in blood pressure based on a first set of values of the plurality of patient parameters representing a present condition of the patient, determine a second predicated change in blood pressure based on a second set of values of the plurality of patient parameters representing an alternative condition of the patient, and provide an absolute or relative indication associated with the first and second predicted changes in blood pressure. In this way, a patient and/or clinician may qualitatively identify potential changes of the patient parameters that may achieve a target change in blood pressure.

In some examples, the indication associated with the predicted change in blood pressure may include further testing suggestions. For example, values of patient parameters for which computational model 226 may receive input may not be complete or may be outdated, such that the values may be zero or based off an estimate or default. Model execution module 234 may identify one or more patient parameters for which missing or outdated values may be further included, and output the indication to include such identified patient parameters. In some examples, model execution module 234 may further identify whether the missing or outdated values may be for patient parameters that are particularly significant for potentially changing the blood pressure, such as described above, and output the indication associated with those significant patient parameters.

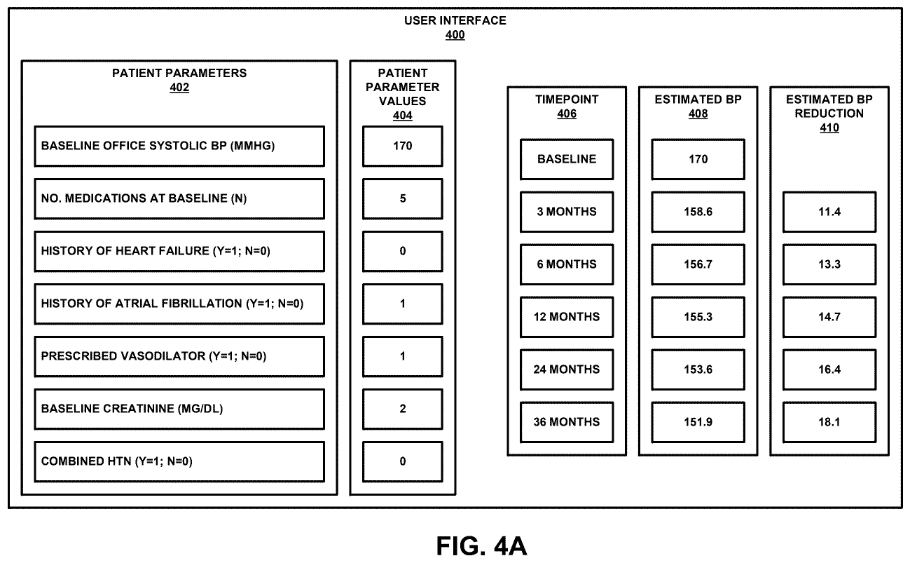

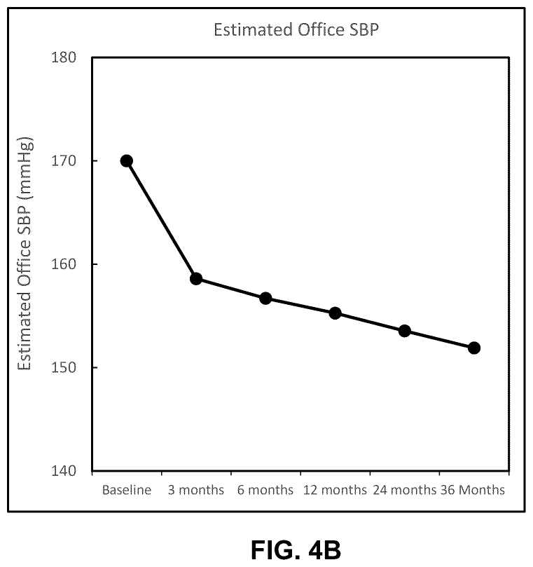

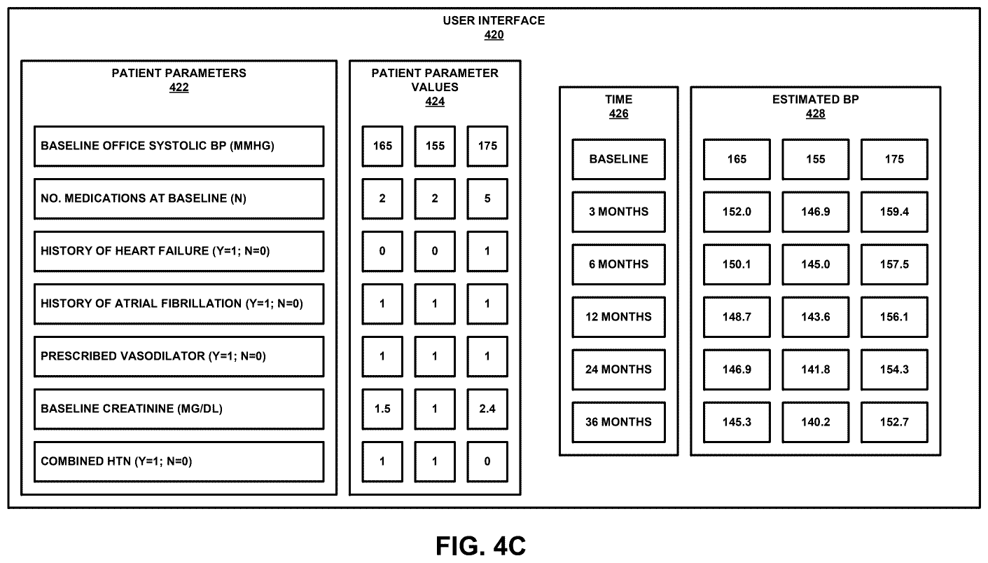

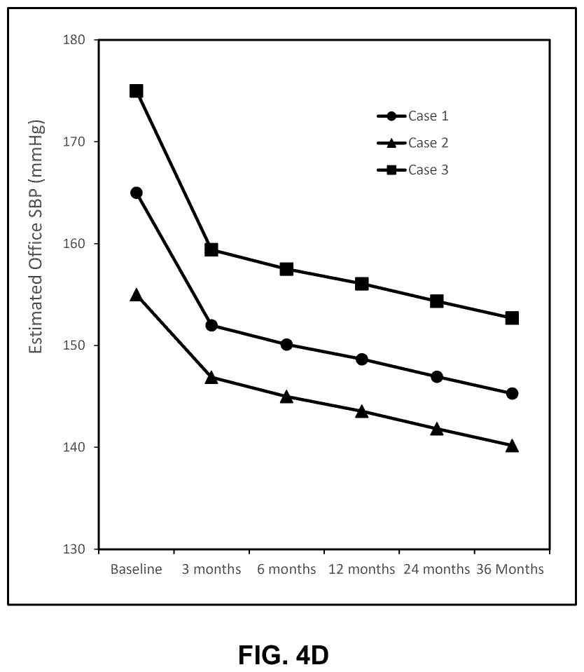

In examples in which computational model 226 accommodates multiple timepoints, model execution module 234 may determine the predicted change in blood pressure of the patient across a plurality of timepoints and output the predicted change in blood pressure of the patient at the plurality of timepoints. For example, population parameter data 224 may include blood pressure entries for various follow-up timepoints after denervation therapy, such that computational model 226 may include one or more variables that account for a time-based reduction for the various timepoints. In some examples, such as will be illustrated in FIGS. 4A-4D below, the indication includes a graph of the predicted change in blood pressure at the plurality of timepoints. Such a visual indication may provide a patient and/or clinician with a potential timeline of blood pressure, which may be particularly useful for patients that may have time-based conditions for which a particular blood pressure may be a target at a future date.

In some examples, model execution module 234 may determine a predicted change in blood pressure on-demand. For example, a patient and/or clinician may operate computing system 12, such as by causing model execution module 234 to obtain the values of the plurality of patient parameters and determine the predicted change in blood pressure, in an office setting to generate the indication and further discuss implications of the predicted change in blood pressure. Such discussion may include risks and costs associated with denervation therapy.

Additionally or alternatively, model execution module 234 may determine a predicted change in blood pressure periodically or in response to a change in patient parameter data 216 and/or computational model 226. In some examples, patient parameter data 216 may be updated with new patient data, such as due to newly-available medical records or based on further testing. In such examples, model execution module 234 may determine the predicted change in blood pressure based on the updated patient parameter data. In some examples, computational model 226 itself may be updated, such as with new values of weighting coefficients and/or bias coefficients as patient parameter data 224 is updated. In such examples, model execution module 234 may determine the predicted change in blood pressure based on the updated computational model 226. In some examples, computational model 226 may be updated with a new selection of inputs of patient parameters, and corresponding new weighting coefficients. In such examples, model execution module 234 may determine the predicted change in blood pressure based on the updated computational model 226 and updated patient parameter data that corresponds to the new or updated weighting coefficients. In any such examples described above, computing system 12 may output an indication, such as to a clinician, of the update, including whether further evaluation may be needed.

While the example techniques of FIG. 3A has been described with respect to regression model 228, in some examples, model execution module 234 may be configured to determine the predicted change in blood pressure using other models, such as machine learning model 230. For example, model execution module 234 may use a neural network that receives an input layer that includes an input vector of the values of the patient parameters. The neural network may include one or more hidden layers that include a plurality of neurons, and for each neuron, computes a weighted sum of inputs based on a weight vector and a bias coefficient, and applies an activation function to the weighted sum. Similarly, the neural network may include one or more output layers that include one or more neurons, and for each neuron, computes a weighted sum of inputs based on a weight vector and bias coefficient.

In some examples, model execution module 234 is configured to execute a computational model 226, such as regression model 228 or machine learning model 230, to predict a different response to denervation therapy. For example, model execution module 230 may predict a physiological parameter other than blood pressure, such as heart rate or an estimated likelihood of another physiological outcome, based on patient parameters. Computational model 226 may include a computational model that receives, as inputs, patient parameters data 216 and generates, based on patient parameters from patient parameter data 216, a predicted alternative physiological parameter or condition that may change or occur in response to denervation therapy. Such a computational model 226 may be generated using population parameter data 224 that includes one or more fixed effects and one or more physiological measurements, such as blood pressure or heart rate, or physiological outcomes, such as myocardial infarction or renal failure.

Referring back to FIG. 2, for computing devices 12 that include model generation module 236, model generation module 236 may be configured to generate computation model 226 based on population parameter data 224. FIG. 3B is a flow diagram illustrating an example technique for generating a mixed multivariate linear regression model as a computational model for estimating a response to denervation therapy. The example technique of FIG. 3B will be described with respect to model generation module 236.

Model generation module 236 may obtain medical records data for a population of patients with hypertension that have undergone denervation therapy (310). For example, model generation module 236 may access population parameter data 224 in memory 202 for the medical records data. The medical records data for each patient in the population may include a value for at least one fixed effect and at least one blood pressure or other physiological measurement or outcome for which a prediction is desired.

In some examples, to determine the plurality of weighting coefficients for the computational model based on the medical records data, particularly medical records that may have varying entries of multivariate data, model generation module 236 may generate a mixed multivariate linear regression model. To generate the mixed multivariate linear regression model, the example technique of FIG. 3B includes fitting a mixed multivariate linear regression model to the medical records data (312), including the fixed effects and the blood pressure measurements of the population of patients. Mixed multivariate linear regression models incorporate both fixed and random effects, and may be fitted to blood pressure measurements, such as office and ambulatory systolic and diastolic blood pressure, as outcome variables. Fixed effects may include any of the plurality of patient parameters of patient parameter data 216, such as baseline blood pressure, prescribed antihypertensive medication classes, number of medications at baseline and over time, and baseline characteristics including age, sex, history of myocardial infarction, type 2 diabetes, body mass index (BMI), sleep apnea, smoking history, heart failure, atrial fibrillation, baseline estimated glomerular filtration rate (eGFR), baseline serum creatinine, procedure duration, catheter time, contrast volume, and number of ablations.

The resulting mixed multivariate linear regression model may include a large number of fixed effects, some of which may not be particularly clinically significant in terms of having minimal impact on the actual estimated result. Model generation module 236 may identify a plurality of significant fixed effects as the plurality of patient parameters (314). In addition to fixed effects, the mixed multivariate linear regression model may include random effects that result from different blood pressure measurements within the same patient, and clinical trials. These nested random effects may be modeled, such as by using an unstructured covariance matrix.

Model generation module 236 may update the mixed multivariate linear regression model with the plurality of patient parameters and fit the updated mixed multivariate linear regression model to define the plurality of weighting coefficients (316). Model generation module 236 may estimate follow-up blood pressure measurements for different timepoints (318). For example, model generation module 236 may use a maximum likelihood approach to estimate the follow-up blood pressure measurements at each follow-up visit.

While the method of FIG. 3B has been described with respect to regression model 228, in some examples, model generation module 236 may be configured to generate machine learning model 230 based on population parameter data 224. Machine learning may generally enable computing system 12 to analyze input data, including population parameters data 224, and identify an action to be performed responsive to the input data. Each machine learning model 230 may be trained using training data that reflects likely input data. The training data may be labeled or unlabeled (meaning that the correct action to be taken based on a sample of training data is explicitly stated or not explicitly stated, respectively). Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

To generate machine learning model 230, model generation module 236 may train a deep learning model to represent a relationship of fixed effects of patients in a population to changes in blood pressure resulting from denervation therapy. For example, model generation module 236 may train the deep leaning model by adjusting the weights of a hidden layer of a neural network model to balance the contribution of each input (e.g., the fixed effects included in population parameter data 224).

Referring back to FIG. 2, processing circuitry 204 may execute user interface module 238, which may display an output of computational model 226 on display 206. FIGS. 4A-4D below are example instances of user interfaces that may be generated as user interface module 238. Each user interface may correspond to a computational model, various inputs received by computational model 226, and various outputs generated for computational model 226. User interface module 238 may generate a particular user interface in response to selection of a particular computational model, such that model execution module 234 may obtain values for one or more patient parameters and/or display an indication associated with the predicted change in blood pressure.

FIG. 4A is a diagram of an example user interface 400 for receiving patient parameters and outputting various indications associated with a predicted change in office systolic blood pressure after denervation therapy. User interface 400 is configured to obtain inputs that include a plurality of patient parameters 402 and patient parameter values 404, and display outputs that include timepoint 406, estimated blood pressure 408 at a particular time point relative to a baseline, and estimated blood pressure reduction 410.

Patient parameters 402 may be populated or selected based on a particular computational model. In the example of FIG. 4A, patient parameters 402 include baseline office systolic blood pressure, number of medications at baseline, history of heart failure, history of atrial fibrillation, prescribed vasodilator, baseline creatinine, and combined hypertension. These patient parameters 402 may be particularly significant for indicating an effectiveness of denervation therapy on blood pressure. Patient parameter values 404 may indicate a value of a particular patient parameter, such as values obtained as input data 218, sensor data 220, and/or medical records data 222. In some examples, patient parameter values 404 may be selectable, such as via input device 210, to receive input data 218.

As described above, model execution module 234 determines, using computational model 226, a predicted change in blood pressure of the patient after denervation therapy based on patient parameter values 404 for various timepoints 406, in which each patient parameter value 404 is modified by a weighting coefficient of computational model 226. While not shown in FIG. 4A, in some examples, user interface 400 may also display values for the weighting coefficients. Model execution module 234 outputs an indication associated with the predicted change in blood pressure of the patient. User interface module 238 displays the indication as timepoint 406, an estimated blood pressure 408 generated from the patient parameter values 404 at a corresponding timepoint 406 based on a baseline blood pressure, and an estimated blood pressure reduction 410 at the corresponding timepoint 406.

FIG. 4B is a graph of an output of estimated blood pressure 408 at timepoints 406 as indicated in example user interface 400 of FIG. 4A. While not shown, in some examples, the graph may include a confidence interval that may reflect uncertainty in the computational model, such as uncertainty associated with random effects or regularized data.

FIG. 4C is a diagram of an example user interface 420 for receiving patient parameters and outputting an indication associated with a predicted change in office systolic blood pressure after denervation therapy for three different use cases. User interface 420 is configured to obtain inputs that include a plurality of patient parameters 422 and patient parameter values 424 for three different cases, and display outputs that include timepoints 426 and estimated blood pressure 428 at a particular time point for the three different cases. The three different cases may include one case of the patient and two cases of either the patient having different patient parameter values or a hypothetical patient representative of a particular population (e.g., a population that shares demographic and medical history with the patient). Such a comparison of cases may enable the patient or a clinician to illustrate how a change in patient parameters values 424, whether through lifestyle changes or medical interventions, may affect blood pressure after denervation therapy.

FIG. 4D is a graph of an output of the example user interface of FIG. 4C, in accordance with some examples of the current disclosure. As shown in FIG. 4D, each case 1, 2, or 3 is plotted with respect to an estimated office systolic blood pressure for the various timepoints.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various aspects of the techniques may enable the following examples.

Example 1: A computing system includes a memory; and one or more processors coupled to the memory, the one or more processors being configured to: obtain values for a plurality of patient parameters of a patient with hypertension, wherein each patient parameter of the plurality of patient parameters relates to a physiological condition of the patient; before delivery of denervation therapy to the patient, determine, using a computational model, a predicted change in blood pressure of the patient if the denervation therapy is delivered based on the values of the plurality of patient parameters; and output, responsive to determining the predicted change in blood pressure of the patient, an indication associated with the predicted change in blood pressure of the patient.

Example 2: The computing system of example 1, wherein the physiological conditions include at least one of a physiological measurement or a medical history event of the patient.

Example 3: The computing system of any of examples 1 and 2, wherein the plurality of patient parameters include at least one of patient demographic characteristics, patient ambulatory blood pressure monitoring (ABPM) characteristics, patient imaging characteristics, patient physiological characteristics, or patient procedural and medication history.

Example 4: The computing system of any of examples 1 through 3, wherein the plurality of patient parameters includes at least one of baseline office systolic blood pressure, number of blood pressure medications at baseline, history of heart failure, history of atrial fibrillation, prescribed vasodilator, baseline creatinine, or combined hypertension.

Example 5: The computing system of any of examples 1 through 4, wherein the one or more processors are configured to: determine the predicted change in blood pressure of the patient for each of a plurality of timepoints; and output the predicted change in blood pressure of the patient for the plurality of timepoints.

Example 6: The computing system of example 5, wherein the indication includes a graph of the predicted change in blood pressure across the plurality of timepoints.

Example 7: The computing system of any of examples 1 through 6, wherein the one or more processors are configured to: determine whether the predicted change in blood pressure exceeds a denervation therapy threshold; determine whether the patient is or is not a candidate for the denervation therapy based on whether the predicted change in blood pressure exceeds the denervation therapy threshold; and output whether the patient is or is not a candidate for the denervation therapy.

Example 8: The computing system of any of examples 1 through 7, wherein each patient parameter of the plurality of patient parameters is modified by a weighting coefficient of a plurality of weighting coefficients of the computational model.

Example 9: The computing system of any of examples 1 through 8, wherein the computational model comprises a mixed multivariate linear regression model.

Example 10: The computing system of any of examples 1 through 9, wherein the computational model comprises a machine learning model.

Example 11: The computing system of any of examples 1 through 10, wherein the computational model is derived from medical records data for a population of patients with hypertension.

Example 12: The computing system of example 11, wherein the one or more processors comprise a first set of one or more processors, and wherein the computing system further comprising a second set of one or more processors configured to generate the computational model.

Example 13: The computing system of example 12, wherein, to generate the computational model, the second set of one or more processors are configured to: obtain the medical records data, wherein the medical records data for each patient in the population includes a value for at least one fixed effect and at least one blood pressure measurement; and determine a plurality of weighting coefficients based on the medical records data for the population of patients.

Example 14: The computing system of example 13, wherein the at least one fixed effect includes at least one of patient demographic characteristics, patient ambulatory blood pressure monitoring (ABPM) or home blood pressure monitoring (HBPM) characteristics, patient imaging characteristics, patient physiological characteristics, or patient procedural and medication history.

Example 15: The computing system of any of examples 13 and 14, wherein, to determine the plurality of weighting coefficients, the one or more processors are configured to: fit a mixed multivariate linear regression model to the fixed effects and the blood pressure measurements of the population of patients; identify a plurality of significant fixed effects as the plurality of patient parameters; and update the mixed multivariate linear regression model with the plurality of patient parameters to define the plurality of weighting coefficients.

Example 16: The computing system of any of examples 1 through 15, wherein to obtain the plurality of patient parameters, the one or more processors are configured to at least one of: receiving, by the computing system and via a user interface, a value for at least one patient parameter of the plurality of patient parameters; or receiving, by the computing system and from a medical records database, a value for at least one patient parameter of the plurality of patient parameters.

Example 17: A method includes obtaining, by a computing system, values for a plurality of patient parameters of a patient with hypertension, wherein each patient parameter of the plurality of patient parameters relates to a physiological condition of the patient; before delivery of denervation therapy to the patient, determining, by the computing system and using a computational model, a predicted change in blood pressure of the patient if the denervation therapy is delivered based on the values of the plurality of patient parameters; and outputting, by the computing system and responsive to determining the predicted change in blood pressure of the patient, an indication associated with the predicted change in blood pressure of the patient.

Example 18: The method of example 17, wherein the physiological condition includes at least one of a physiological measurement or a medical history event of the patient.

Example 19: The method of any of examples 17 and 18, wherein the plurality of patient parameters includes at least one of patient demographic characteristics, patient ambulatory blood pressure monitoring (ABPM) or home blood pressure monitoring (HBPM) characteristics, patient imaging characteristics, patient physiological characteristics, or patient procedural and medication history.

Example 20: The method of any of examples 17 through 19, wherein the plurality of patient parameters includes at least one of baseline office systolic blood pressure, number of medications at baseline, history of heart failure, history of atrial fibrillation, prescribed vasodilator, baseline creatinine, or combined hypertension.

Example 21: The method of any of examples 17 through 20, wherein determining the predicted change in blood pressure of the patient comprises determining, by the computing system, the predicted change in blood pressure of the patient for each of a plurality of timepoints, and wherein outputting the predicted change in blood pressure of the patient comprises outputting, by the computing system, the predicted change in blood pressure of the patient for the plurality of timepoints.

Example 22: The method of example 21, wherein the indication includes a graph of the predicted change in blood pressure across the plurality of timepoints.

Example 23: The method of any of examples 17 through 22, further includes determining, by the computing system, whether the predicted change in blood pressure exceeds a denervation therapy threshold; determining, by the computing system, whether the patient is or is not a candidate for the denervation therapy based on whether the predicted change in blood pressure exceeds the denervation therapy threshold; and outputting, by the computing system, whether the patient is or is not a candidate for the denervation therapy.

Example 24: The method of any of examples 17 through 23, wherein each patient parameter of the plurality of patient parameters is modified by a weighting coefficient of a plurality of weighting coefficients of the computational model.

Example 25: The method of any of examples 17 through 24, wherein the computational model comprises a mixed multivariate linear regression model.

Example 26: The method of any of examples 17 through 25, wherein the computational model comprises a machine learning model.

Example 27: The method of any of examples 17 through 26, wherein the computational model is derived from medical records data for a population of patients with hypertension.

Example 28: The method of example 27, further comprising determining, by the computing system, the computational model.

Example 29: The method of example 28, wherein determining the computational model further comprises: obtaining, by the computing system, the medical records data, wherein the medical records data for each patient in the population includes a value for at least one fixed effect and at least one blood pressure measurement; and determining, by the computing system, a plurality of weighting coefficients based on the medical records data for the population of patients.

Example 30: The method of example 29, wherein the at least one fixed effect includes at least one of patient demographic characteristics, patient ambulatory blood pressure monitoring (ABPM) characteristics, patient imaging characteristics, patient physiological characteristics, or patient procedural and medication history.

Example 31: The method of any of examples 29 and 30, wherein determining the plurality of weighting coefficients further comprises: fitting, by the computing system, a mixed multivariate linear regression model to the fixed effects of and the blood pressure measurements of the population of patients; identifying, by the computing system, a plurality of significant fixed effects as the plurality of patient parameters; updating, by the computing system, the mixed multivariate linear regression model with the plurality of patient parameters to define the plurality of weighting coefficients.

Example 32: The method of any of examples 17 through 31, wherein obtaining the plurality of patient parameters comprises at least one of: receiving, by the computing system and via a user interface, a value for at least one patient parameter of the plurality of patient parameters; or receiving, by the computing system and from a medical records database, a value for at least one patient parameter of the plurality of patient parameters.

Various examples have been described. These and other examples are within the scope of the following claims.

What is claimed is:

1. A computing system comprising:
a memory; and
one or more processors coupled to the memory, the one or more processors being configured to:
obtain values for a plurality of patient parameters of a patient with hypertension, wherein each patient parameter of the plurality of patient parameters relates to a physiological condition of the patient;
before delivery of denervation therapy to the patient, determine, using a computational model, a predicted change in blood pressure of the patient if the denervation therapy is delivered based on the values of the plurality of patient parameters; and
output, responsive to determining the predicted change in blood pressure of the patient, an indication associated with the predicted change in blood pressure of the patient.

2. The computing system of claim 1, wherein the physiological conditions include at least one of a physiological measurement or a medical history event of the patient.

3. The computing system of claim 1, wherein the plurality of patient parameters include at least one of patient demographic characteristics, patient ambulatory blood pressure monitoring (ABPM) characteristics, patient imaging characteristics, patient physiological characteristics, or patient procedural and medication history.

4. The computing system of claim 1, wherein the plurality of patient parameters includes at least one of baseline office systolic blood pressure, number of blood pressure medications at baseline, history of heart failure, history of atrial fibrillation, prescribed vasodilator, baseline creatinine, or combined hypertension.

5. The computing system of claim 1, wherein the one or more processors are configured to:
determine the predicted change in blood pressure of the patient for each of a plurality of timepoints; and
output the predicted change in blood pressure of the patient for the plurality of timepoints.

6. The computing system of claim 5, wherein the indication includes a graph of the predicted change in blood pressure across the plurality of timepoints.

7. The computing system of claim 1, wherein the one or more processors are configured to:
determine whether the predicted change in blood pressure exceeds a denervation therapy threshold;
determine whether the patient is or is not a candidate for the denervation therapy based on whether the predicted change in blood pressure exceeds the denervation therapy threshold; and
output whether the patient is or is not a candidate for the denervation therapy.

8. The computing system of claim 1, wherein each patient parameter of the plurality of patient parameters is modified by a weighting coefficient of a plurality of weighting coefficients of the computational model.

9. The computing system of claim 1, wherein the computational model comprises a mixed multivariate linear regression model.

10. The computing system of claim 1, wherein the computational model comprises a machine learning model.

11. The computing system of claim 1, wherein the computational model is derived from medical records data for a population of patients with hypertension.

12. The computing system of claim 11,
wherein the one or more processors comprise a first set of one or more processors, and
wherein the computing system further comprising a second set of one or more processors configured to generate the computational model.

13. The computing system of claim 12, wherein, to generate the computational model, the second set of one or more processors are configured to:
obtain the medical records data, wherein the medical records data for each patient in the population includes a value for at least one fixed effect and at least one blood pressure measurement; and
determine a plurality of weighting coefficients based on the medical records data for the population of patients.

14. The computing system of claim 13, wherein the at least one fixed effect includes at least one of patient demographic characteristics, patient ambulatory blood pressure monitoring (ABPM) or home blood pressure monitoring (HBPM) characteristics, patient imaging characteristics, patient physiological characteristics, or patient procedural and medication history.

15. The computing system of claim 13, wherein, to determine the plurality of weighting coefficients, the one or more processors are configured to:
fit a mixed multivariate linear regression model to the fixed effects and the blood pressure measurements of the population of patients;
identify a plurality of significant fixed effects as the plurality of patient parameters; and
update the mixed multivariate linear regression model with the plurality of patient parameters to define the plurality of weighting coefficients.

16. The computing system of claim 1, wherein to obtain the plurality of patient parameters, the one or more processors are configured to at least one of:
receiving, by the computing system and via a user interface, a value for at least one patient parameter of the plurality of patient parameters; or
receiving, by the computing system and from a medical records database, a value for at least one patient parameter of the plurality of patient parameters.

17. A method, comprising:
obtaining, by a computing system, values for a plurality of patient parameters of a patient with hypertension, wherein each patient parameter of the plurality of patient parameters relates to a physiological condition of the patient;
before delivery of denervation therapy to the patient, determining, by the computing system and using a computational model, a predicted change in blood pressure of the patient if the denervation therapy is delivered based on the values of the plurality of patient parameters; and
outputting, by the computing system and responsive to determining the predicted change in blood pressure of the patient, an indication associated with the predicted change in blood pressure of the patient.

18. The method of claim 17, further comprising:
determining, by the computing system, whether the predicted change in blood pressure exceeds a denervation therapy threshold;
determining, by the computing system, whether the patient is or is not a candidate for the denervation therapy based on whether the predicted change in blood pressure exceeds the denervation therapy threshold; and
outputting, by the computing system, whether the patient is or is not a candidate for the denervation therapy.

19. The method of claim 17, wherein the computational model is derived from medical records data for a population of patients with hypertension.

20. The method of claim 17, further comprising generating, by the computing system, the computational model, wherein generating the computational model comprises:
obtaining, by the computing system, the medical records data, wherein the medical records data for each patient in the population includes a value for at least one fixed effect and at least one blood pressure measurement; and
determining, by the computing system, a plurality of weighting coefficients based on the medical records data for the population of patients.
